(19) United States
(12) Patent Application Publication
(10) Pub. No.: US 2007/0099988 A1
(43) Pub. Date: May 3, 2007

(54) ANTI-EMETIC USES OF (3R, 4R)-DELTA8-TETRAHYDROCANNABINOL-11-OIC ACIDS

(75) Inventor: Bobby W. Sandage JR., Lexington, MA (US)

Correspondence Address: FOLEY AND LARDNER LLP;SUITE 500, 3000 K STREET NW, WASHINGTON, DC, 20007, US

(73) Assignee: Indevus Pharmaceuticals, Inc.

(21) Appl. No.: 11/534,149

(22) Filed: Sep. 21, 2006

Related U.S. Application Data
(60) Provisional application No. 60/719,205, filed on Oct. 31, 2005.

(51) Int. Cl. A61K 31/353

(52) U.S. Cl. 514/454

(57) ABSTRACT

The present invention relates to non-psychoactive derivatives of tetrahydrocannabinol, (3R,4R)-Δ⁸-THC-11-oic acids, for treating or preventing nausea and relieving symptoms thereof.

### CROSS-REFERENCE TO RELATED PATENT APPLICATIONS

This application claims priority to U.S. provisional application No. 60/719,205 filed Oct. 31, 2005 the disclosure of which has been incorporated by reference herein in its entirety.

### FIELD OF THE INVENTION

The present invention relates to the treatment of nausea using non-psychoactive derivatives of tetrahydrocannabinol, which exhibit anti-inflammatory and analgesic properties. In particular, the present invention further relates to the use of (3R,4R)-Δ⁸-tetrahydrocannabinol-11-oic acids, and pharmaceutical compositions comprising therapeutically effective amounts of the acids, for the treatment of nausea.

### BACKGROUND OF THE INVENTION

1. THC Derivatives

Δ⁹-Tetrahydrocannabinol (THC]), is the major psychoactive constituent of marijuana. 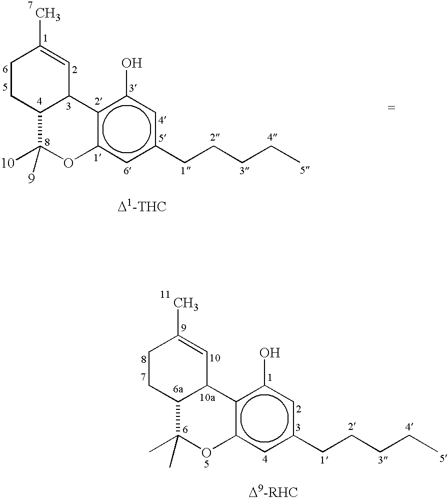
In addition to mood-altering effects, THC exhibits other activities which may have therapeutic value. The potential therapeutic value of THC has led to a search for related compounds which, while devoid of psychoactive effects, retain the activities of potential medicinal value.

Previous work with Δ⁸-tetrahydrocannabinol [(3R,4R) 6a,7,10,10a-tetrahydro-6,6,9-trimethyl-3-pentyl-6H-dibenzo[b,d]pyran-1-ol, hereinafter referred to as Δ⁸-THC], which is depicted below in Formula II, has indicated that certain derivatives of this compound may prove clinically useful. 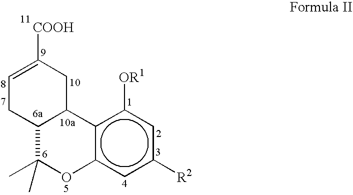
The 11-carboxy derivative of Δ⁸-THC [Δ⁸-THC-11-oic acid] has been reported to be a non-psychoactive, potent antagonist to endogenous platelet activating factor and, thus, a useful treatment for PAF-induced disorders, such as asthma, systemic anaphylaxis, and septic shock. (See U.S. Pat. No. 4,973,603, incorporated herein by reference.) Another derivative, (3S,4S)-11-hydroxy-Δ⁸-THC-1′,1′ dimethylheptyl, essentially free of the (3R,4R) form, has been reported to possess analgesic and anti-emetic activities. (See U.S. Pat. No. 4,876,276, also incorporated herein by reference.)
2. Nausea

Nausea and vomiting occur for many reasons. The most common causes are motion sickness, which may occur in many settings including travel by car, air, or boat, and can last a few hours to a few days. Viral infections can cause nausea and vomiting, as can food poisoning More than 250 different diseases can cause food poisoning. The most common diseases are infections caused by bacteria, such as campylobacter, salmonella, shigella, E. coli, listeria and botulism.

Some medications can cause nausea, such as anti-cancer drugs and morphine, as well as radiation therapy for cancer.

Many antiemetics have been developed to decrease the severity of nausea. Meclizine hydrochloride (Bonine®) is an antihistamine that is effective in the treatment of nausea, vomiting, and dizziness associated with motion sickness. It should not be taken by people with lung diseases, glaucoma, or difficulty with urination due to an enlarged prostate unless recommended by a physician. Meclizine may cause drowsiness and should not be taken with other medicines having sedative side effects such as alcohol, tranquilizers, or sleeping pills. Due to drowsiness, persons using meclizine should not drive or operate dangerous machinery. Meclizine is not recommended in children under 12 or in pregnant or nursing females unless recommended by a doctor.

Dimenhydrinate (Dramamine®) also is an antihistamine. Its use should be limited to motion sickness. Due to the potential for causing drowsiness, dimenhydrinate should be avoided in the same situations as Meclizine.

Emetrol® is an oral solution designed to soothe the stomach when nausea and vomiting are caused by a viral or bacterial infection or overeating. Emetrol contains sugar and phosphoric acid. Diabetics should not use Emetrol without medical supervision because of the concentrated sugar. According to its manufacturer, Emetrol should not be taken for more than five doses in one hour without consulting a physician. A doctor should also be consulted when considering using this medicine for pregnant or nursing women and young children.

None of the currently used treatments for nausea is capable of fully relieving the symptoms in all cases. Patients frequently combine different treatments in an attempt to address all of their symptoms. Clearly, although numerous treatments have been developed in an attempt to control nausea there is still a great need in the art for effective treatments.

3. Description of Related Art

U.S. Pat. No. 5,338,753 discloses (3R,4R)-Δ⁶-THC-7-oic acids (which correspond to (3R,4R)-Δ⁸-THC-11-oic acids, but were named using an alternative numbering system) that are useful as anti-inflammatory agents and analgesics, as well as methods of synthesizing them, but does not disclose compositions or methods for treating patients suffering from nausea.

U.S. Pat. Nos. 6,162,829 and 6,355,650 disclose derivatives of (3R,4R)-Δ⁸-THC-11-oic acids that are also useful as anti-inflammatory agents and analgesics, and methods of synthesizing them. They do not disclose compositions or methods for treating patients suffering from nausea.

U.S. Pat. No. 6,448,288 discloses the use of Δ⁸-THC-11-oic acids to decrease cell proliferation, but fails to disclose compositions or methods for treating patients suffering from nausea.

U.S. Published Application No. 2004/0054007 discloses methods for decreasing cell proliferation using (3R,4R)-Δ⁸-THC-11-oic acid, but it also fails to disclose compositions or methods for treating patients suffering from nausea.

U.S. Published Application No. 2004/0225011 discloses methods of using cannabinoid compounds that are derivatives of THC to decrease cell proliferation, and does not disclose compositions or methods for treating patients suffering from nausea.

The disclosures of each of these patents and published applications are incorporated herein by reference in their entirety.

It is desired, however, to provide a method of treating, alleviating, and/or relieving symptoms associated with nausea by use of the Δ⁸-THC-11-oic acid derivatives, such as those described above, as well as pharmaceutical compositions suitable for such use.

### SUMMARY OF THE INVENTION

An object of the present invention is to provide compositions and methods for treating a patient suffering from nausea, whereby the (3R,4R)-Δ⁸-THC-11-oic acids are administered to said patient.

According to a first aspect of the present invention, unique methods are provided for the treatment of nausea in a mammal using a compound having Formula II 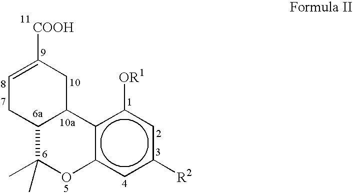
wherein R¹ is hydrogen, —COCH₃ or —COCH₂CH₃; and R² is a branched C₅-C₁₂ alkyl group which may optionally have a terminal aromatic ring, or optionally a branched —OCHCH₃(CH₂)ₘ alkyl group which may have a terminal aromatic ring, wherein m is 0 to 7. The method comprises the steps of identifying a mammal suffering from or suspected of suffering from nausea; and administering to the mammal an effective amount of the compound of formula II, or a pharmaceutically acceptable salt, ester, or solvate thereof.

According to a second aspect of the invention, compositions are provided for use in treating nausea in a mammal, particularly humans, including a therapeutically effective amount of a compound having Formula II 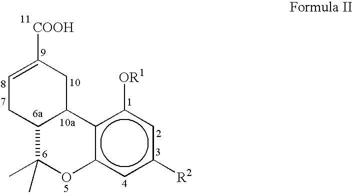
wherein R¹ is hydrogen, —COCH₃ or —COCH₂CH₃; and R² is a branched C₅-C₁₂ alkyl group, which may optionally have a terminal aromatic ring, or optionally a branched —OCHCH₃(CH₂)ₘ alkyl group, which may have a terminal aromatic ring, wherein m is 0 to 7, or a pharmaceutically acceptable salt, ester or solvate thereof.

The pharmaceutical composition may optionally include a therapeutically effective amount of one or more compounds selected from the group consisting of sodium pentosanpolysulfate, antihistamines, antidepressants, imipramine, antispasmodics, urinary anesthetics, and capsaicin.

The pharmaceutical composition may also include a therapeutically effective amount of an anticholinergic agent selected from the group consisting of anisotropine, aprophen, artane, atropine, belladonna, benactyzine, benztropine, clidinium, dicyclomine, glycopyrrolate, homatropine, hyoscyamine, isopropamide, mepenzolate, methantheline, methscopolamine, oxybutynin, oxyphencyclimine, propantheline, scopolamine, terodiline, tridihexethyl, trihexyphenidyl, and trospium.

According to a third aspect of the invention, unique compositions and methods are provided for use in treating nausea in a mammal, including an effective amount of a compound having Formula III (IP-571) 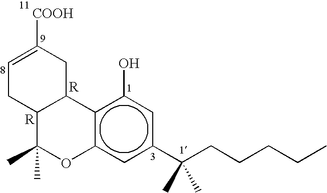
or a pharmaceutically acceptable salt, ester or solvate thereof.

### BRIEF DESCRIPTION OF THE DRAWINGS

FIG. 1 is a synthetic scheme for the (3R,4R)-Δ⁸-THC-11-oic acids of the present invention.

### DETAILED DESCRIPTION OF THE PREFERRED EMBODIMENTS

### 1. Introduction

The present invention relates to (3R,4R)-Δ⁸-THC-11-oic acids, pharmaceutical compositions comprising therapeutically effective amounts of these compounds, and methods of treating nausea in a mammal by administering such compounds or pharmaceutical compositions. The THC derivatives of the present invention have reduced or no psychoactivity and do not bind to the CB1 receptor. An exemplary Δ⁸-THC-11-oic acid analog in accordance with the present invention is 1′,1′-dimethylheptyl-Δ⁸-THC-11-oic acid (IP-571), also known as CT3 or ajulemic acid, shown below in Formula III:

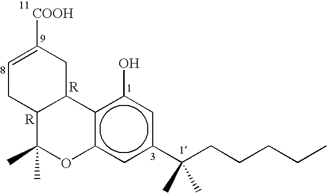

### 2. Compositions

The present invention relates to compositions and pharmaceuticals useful in relieving symptoms of nausea, which comprise Δ⁸-THC-11-oic acids and derivatives and analogs thereof, as depicted below in Formula II: 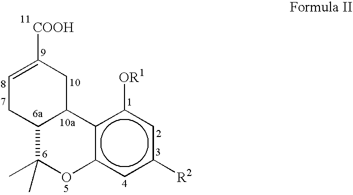
wherein R¹ is hydrogen, —COCH₃ or —COCH₂CH₃; R² is a branched C₅-C₁₂ alkyl compound, which may optionally have a terminal aromatic ring, or optionally a branched —OCHCH₃(CH₂)ₘ alkyl compound, which may have a terminal aromatic ring, wherein m is 0 to 7.

In one embodiment, R¹ is hydrogen and R² is 1′,1′-dimethylheptyl. Thus, in this form, the compounds have the formula shown in Formula III below:

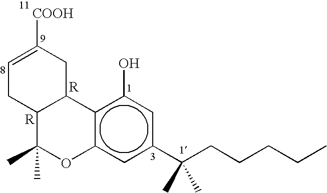

The phrase “therapeutically effective amount” means that amount of the pharmaceutical composition that provides a therapeutic benefit in the treatment, prevention, or management of nausea.

Dosage amounts for the (3R,4R)-Δ⁸-THC-11-oic acids according to the present invention, when administered orally or intravenously (IV) for the relief of symptoms of nausea, are generally between about 1 mg and about 200 mg, for example between about 10 mg and about 100 mg per day, or between about 20 mg and about 60 mg per day, administered about 1, 2, 3 or 4 times daily. As would be understood by one skilled in the art, the dose, and dose frequency, will vary according to the patient's age, body weight, and therapeutic response, as well as the severity of the condition. Typically at a dose of 0.1 mg/kg to 10 mg/kg body weight, for example about 1 mg/kg is given.

The compositions of the present invention may be optionally administered in conjunction with other existing treatments for nausea, including, but not limited to, meclizine, Emetrol®, antihistamines such as hydroxizine (Atarax® Vistaril®) and dimenhydrinate.

The orally administered compounds and pharmaceutical compositions according to the present invention may be optionally administered in conjunction with existing treatments for nausea that are administered via IV.

According to one embodiment of the invention, the compositions may optionally be administered in conjunction with an anticholinergic agent to inhibit the transmission of parasympathetic nerve impulses and thereby reduce spasms of smooth muscle. The anticholinergic agent can be administered orally or via IV, although other routes of administration are contemplated. Such anticholinergic agents may be selected from anisotropine, aprophen, artane, atropine, belladonna, benactyzine, benztropine, clidinium, dicyclomine, glycopyrrolate, homatropine, hyoscyamine, isopropamide, mepenzolate, methantheline, methscopolamine, oxybutynin, oxyphencyclimine, propantheline, scopolamine, terodiline, tridihexethyl, trihexyphenidyl, and trospium.

The pharmaceutical compositions of the present invention may include the active ingredients described above and pharmaceutically acceptable carriers, excipients and the like, and optionally, other therapeutic ingredients. For instance, the drug may be suspended in a vegetable oil, such as olive oil or peanut oil, and, optionally encapsulated in a gelatin capsule. For human therapy, the compounds or pharmaceutical compositions can be administered orally, in the form of a gelatin capsule, or by IV in the form of a suspension or solution.

The term “pharmaceutically acceptable salt” refers to a salt prepared from pharmaceutically acceptable non-toxic acids or bases, including inorganic and organic compounds. Exemplary salts of Formula II include sodium, potassium and ammonium.

Examples of inorganic bases, for potential salt formation include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine. The compounds and pharmaceutical compositions of the present invention may be administered in the form of such pharmaceutically acceptable salts.

The compounds of interest may also be administered in the form of esters, e.g., methyl, ethyl and the like. Solvates of the compounds of interest may also be useful, including hydrates and the like. Examples of inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic.

The compounds of the present invention may also be included in formulations such as suspensions, solutions and elixirs; aerosols; or carriers such as starches, sugars, microcrystalline cellulose, diluents, granulating agents, lubricants, binders, disintegrating agents, and the like, in the case of oral solid preparations (such as powders, capsules, and tablets), with oral solid preparations being exemplary, such as capsules.

Because of their ease of administration, tablets and capsules represent the most advantageous oral dosage unit form, in which case solid pharmaceutical carriers are employed. If desired, tablets may be coated by standard aqueous or nonaqueous techniques. Because of the benefits of IV for relieving symptoms of nausea, IV formulations are another exemplary dosage form, in which case the compounds and pharmaceutical compositions of the present invention are provided dissolved or suspended in a pharmaceutically acceptable solvent or diluent.

In addition to the dosage forms set out above, the compounds and pharmaceuticals of the present invention may also be administered by controlled release means and/or delivery devices such as those described in U.S. Pat. Nos. 3,845,770; 3,916,899; 3,536,809; 3,598,123; and 4,008,719, the disclosures of which are hereby incorporated by reference.

### 3. Methods

The compounds and pharmaceutical compositions of the present invention can be used in methods of treating mammals suffering from nausea, in both veterinary medicine and human therapy contexts. The method of administering the compounds and pharmaceutical compositions in the acute or chronic management of nausea will vary with the severity of the condition and the route of administration. The dose, and perhaps the dose frequency, will also vary according to the age, body weight, and response of the individual patient. The actual amounts of the active ingredients administered will vary with each case, according to the species of mammal, the nature and severity of affliction being treated, and the method of administration.

The compounds may be administered via any appropriate route, e.g. intravenously, intraarterially, topically, by injection, intraperitoneally, intrapleurally, orally, subcutaneously, intramuscularly, sublingually, intraepidermally, or rectally. Illustrative methods of administration are orally and via IV. The oral formulations may be solutions, suspensions, suppositories, tablets, granules, powders, capsules, ointments, or creams. The IV formulations may be solutions or suspensions, including compositions comprising liposomes. In the preparation of the pharmaceuticals, a solvent (e.g., water or physiological saline), solubilizing agent (e.g., ethanol, Polysorbates, or Cremophor EL7), agent for making isotonicity, preservative, antioxidizing agent, excipient (e.g., lactose, starch, crystalline cellulose, mannitol, maltose, calcium hydrogen phosphate, light silicic acid anhydride, or calcium carbonate), binder (e.g., starch, polyvinylpyrrolidone, hydroxypropyl cellulose, ethyl cellulose, carboxy methyl cellulose, or gum arabic), lubricant (e.g., magnesium stearate, talc, or hardened oils), or stabilizer (e.g., lactose, mannitol, maltose, polysorbates, macrogols, or polyoxyethylene hardened castor oils) can be added. If necessary, glycerin, dimethylacetamide, 70% sodium lactate, a surfactant, or a basic substance such as sodium hydroxide, ethylenediamine, ethanolamine, sodium bicarbonate, arginine, meglumine, or trisaminomethane is added. Pharmaceutical preparations such as solutions, tablets granules or capsules can be formed with these components. Compositions for slow release of the compound can be formed as described in U.S. Pat. No. 4,880,830.

Generally, the oral administration methods and formulations of the present invention provide between about 1 mg and about 200 mg per day, for example between about 10 mg and about 100 mg per day, or between about 20 mg and about 60 mg per day, administered about 2 to about 4 times daily, of the (3R,4R)-Δ⁸-THC-11-oic acids (i.e., excluding excipients, carriers, and any of the optional additional active ingredients described herein). If desired, the daily dose may include two or more unit doses, i.e., tablets, cachets or capsules, to be administered each day.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient response.

The methods of the present invention envision the optional inclusion of existing treatments for nausea in conjunction with the methods of administration and formulation of compounds and pharmaceutical compositions comprising the (3R,4R)-Δ⁸-THC-11-oic acids of the present invention.

Pharmaceutical compositions for use in the methods of the present invention suitable for oral administration may be presented as discrete units such as capsules, cachets, or tablets, or aerosol sprays, each containing a predetermined amount of the active ingredient, as a powder or granules, as creams, pastes, gels, or ointments, or as a solution or a suspension in an aqueous liquid, a non-aqueous liquid, an oil-in-water emulsion, or a water-in-oil liquid emulsion. Formulations that include micelles are also contemplated. Such compositions may be prepared by any of the methods of pharmacy, but all methods include the step of bringing into association the carrier with the active ingredient which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally, with one or more accessory ingredients. Compressed tablets may be prepared by compressing in a suitable machine the active ingredient in a free-flowing form, such as powder or granules, optionally mixed with a binder (e.g., carboxymethylcellulose, gum arabic, gelatin), filler (e.g., lactose), adjuvant, flavoring agent, coloring agent, lubricant, inert diluent, coating material (e.g., wax or plasticizer), and a surface active or dispersing agent. Molded tablets may be made by molding, in a suitable machine, a mixture of the powdered compound moistened with an inert liquid diluent. Those skilled in the art will know, or will be able to ascertain with no more than routine experimentation, appropriate pharmacological carriers for said pharmaceutical compositions.

When the compositions and pharmaceuticals according to the present invention are administered using the IV method, they can be provided as dispersions, suspensions, or solutions.

The methods of the present invention include the determination of optimum doses of the compounds and pharmaceutical compositions for treating nausea symptoms, which may be determined in consideration of the results of animal experiments. More specific doses obviously vary depending on the administration method, the condition of the subject such as age, body weight, sex, sensitivity, food eaten, dosage intervals, medicines administered in combination, and the seriousness and degree of the nausea. The optimal dose and the administration frequency under a given condition must be determined by the appropriate dosage test of a medical specialist based on the aforementioned guidelines, and does not constitute undue experimentation for one skilled in the art.

### 4. Examples

The invention is further defined by reference to the following examples describing in detail the preparation of the compounds and compositions for treating nausea according to the present invention The examples are representative and should not be construed to limit the scope of the invention.

a. Preparation of Derivatives

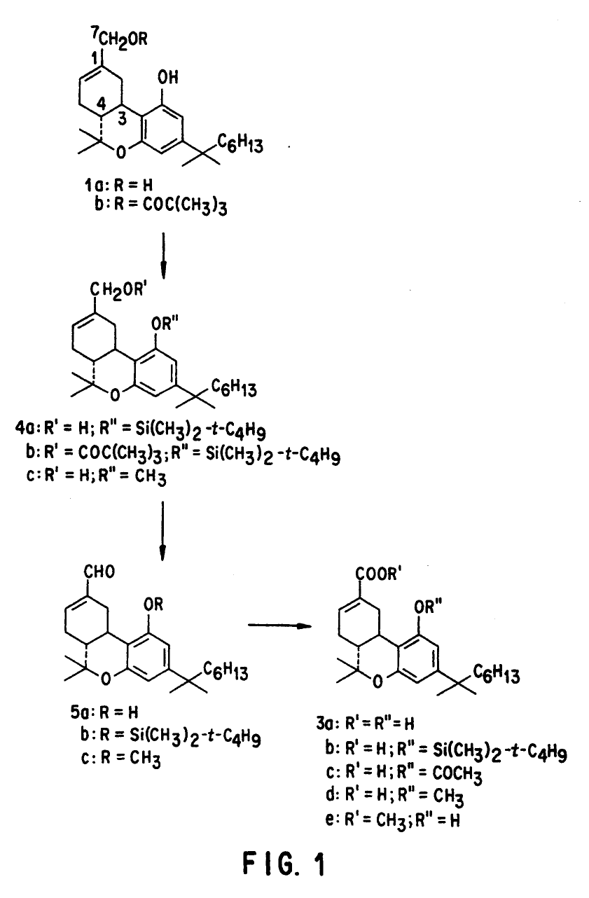

The compounds of the present invention may be prepared according to the synthetic scheme depicted in FIG. 1.

In general, melting points were taken in glass capillary tubes with a Thomas-Hoover Uni-Melt apparatus. Infrared spectra were recorded on a JASCO A-200 spectrophotometer. Rotations were determined on a Perkin-Elmer Model 141 polarimeter in chloroform. Chromatographic separations were performed on silica gel columns (Woelm TSC silica, for dry chromatography, activity III/30 mm, No. 04530). The high-resolution mass spectrometry (HRMS was performed on a Varian 711 instrument.

b. Synthesis of Compound 4a

In general, this esterification follows the procedure of Corey and Venkateswarlu (Corey E. J. and Venkateswarlu A. “Protection of Hydroxyl Groups as Tert-Butyldimethylsilyl Derivatives.” J. Am. Chem. Soc. 94:6190 (1972). Compound 1b (2.9 g, 6.17 mmol), [V]D 1152.60 (c 17.2 m/mL, CHCl₃), prepared according to Mechoulam et al. (Mechoulam R. et al. “Synthesis of the Individual, Pharmacologically Distinct, Enantiomers of a Tetrahydrocannabinol Derivative.” Tetrahydron:Asymmetry 1:315 (1990)), was dissolved in dry dimethylformamide (DMF) (6 mL). Dimethyl-tert-butylsilyl chloride (1.85 g, 12.27 mmol) and imidazole (1.67 g, 24.6 mmol) were added, and the resulting mixture was stirred for 48 hours at 38° C. Water (30 mL) was added, and the mixture was extracted with ether. After evaporation of the dried ether layer, an oil (4b, 3.6 g) was obtained: [∀]_{D} 153° (c 24.45 mg/mL, CHCl₃); IR 8ₘₐₓ (neat) 1725 cm⁻¹, no free hydroxyl groups were observed; ¹H NMR (CDCl₃) Δ 3.28 (1H, br d, J=16 Hz, C-2 eq H), 4.46 (2H, s, C-7 H), 5.70 (1H, m, C-6H), 6.38 (1H, d, J=1.5 Hz, arom), 6.42 (1H, d, J=1.5 Hz, atom). This oil (compound 4b) was used in the next step with no further purification.

A solution of compound 4b (3.2 g, 5.5 mmol) in dry ether (50 mL) was added under a nitrogen atmosphere to lithium aluminum hydride (870 mg) in dry ether (60 mL). The resulting mixture was boiled under reflux for 1.5 hours. After the standard workup (ethyl acetate followed by slow addition of a saturated solution of magnesium sulfate until a clear supernatant was formed), the ether layer was dried and evaporated to give an oil (3.2 g). The oil was chromatographed on a silica gel column (100 g), using ether-petroleum ether (6:4) as eluent, to give the alcohol 4a (8 g 67%): [∀]_{D} −175° (c 7.6 mg/M1, CHCl₃); IR 8ₘₐₓ (neat) 3320 cm⁻¹ (OH band), no carbonyl bands; ¹H NMR (CDCl₃) Δ 3.38 (1H, br d, J=16 Hz, C-2 eq H), 4.02 (2H, s, C-7H), 5.72 (1H, br d, J=16 Hz, C-2 eq H), 4.02 (2H, s, C-7H), 5.72 (1H, br d, C-6 H), 6.36, 6.42 (2H, s, atom).

c. Synthesis of Compound 5b

Following the procedure of Corey and Samuelsson (Corey E. J. and Samuelsson B. “One Step Conversion of Primary Alcohols in the Carbohydrate Series to the Corresponding Carboxylic-Tert-Butyl Esters.” J. Org. Chem. 49:4735 (1984)), dry pyridine (2.3 mL) followed by chromic oxide (1.44 g, 14.4 mmol) was added to a solution of methylene chloride-DMF (4:1) (36 mL). The mixture was stirred for fifteen (15) minutes. The primary allylic hydroxy compound 4a (1.8 g, 3.6 mmol) in methylene chloride-DMF (4:1) (7.2 mL) was added, and the reaction mixture was stirred at room temperature for one (1) hour. Ethanol (1.8 mL) was added, and the mixture was stirred for an additional ten (10) minutes and was then diluted with ethyl acetate (180 mL). The resulting mixture was filtered through a sintered-glass funnel, packed with silica (3 cm), with a layer of anhydrous sodium sulfate on top, and eluted with ethyl acetate (ca 600 mL). The ethyl acetate filtrate was washed with dilute hydrochloric acid (1 N) and then with sodium bicarbonate solution and water. After evaporation of the dried organic solvent, a semisolid compound (5b, 1.7 g, 95%) was obtained. Crystallization from pentane gave the aldehyde 5b: mp 80°-81° C.; [∀]_{D} −268° (c 6.82 mg/mL, CHCl₃), IR 8ₘₐₓ 1690 cm⁻¹ (neat); ¹H NMR (CDCl₃) Δ 3.82 (1 H, br d, J=15 Hz, C-2 eq H), 6.38 and 6.42 (2H, s, atom), 6.80 (1H, m, C-6H), 9.50 (1H, s, C-7H). Anal. (C₃₁H₅₀O₃Si) C, H.

d. Synthesis of (3R,4R)-Δ⁸-THC-DMH-11-oic Acid (3a)

Following the procedure described by Pellegata et al. (Pellegata R et al. “An Improved Procedure for the Synthesis of Oleuropeic Acid.” Synth. Commun. 15:165 (1985)), sodium chloride (488 mg) was added portionwise with vigorous stirring to a mixture of the aldehyde 5b (498 mg, 1 mmol), 2-methyl2-butene (2.24 mL), saturated aqueous potassium dihydrogen phosphate (1.34 mL), and tert-butyl alcohol (22 mL). The reaction mixture was stirred at room temperature for five (5) hours. Water (20 mL) was added, and the mixture was extracted several times with ethyl acetate, dried, and evaporated to give the crude acid which was purified on a silica gel column (10 g, elution with 10% ether-petroleum ether) to give the acid 3b (460 mg, 89%) as an oil: [∀]_{D} −218° (c 13.7 mg/mL, CHCl₃); IR 8ₘₐₓ 1680 cm⁻¹ and a broad band in the 2800-3600 cm⁻¹ region, ¹H NMR (CDCl₃) Δ 3.75 (1H, br d, J=18 Hz, C-2 eq H), 6.23 (1H, d, J=1.5 Hz, arom), 6.27 (1H, d, J=1.5 Hz, arom), 7.00 (1 H, br d, C-6H).

Tetrabutylammonium fluoride (0.6 mmol from a 1.0M solution in THF, Aldrich,) was added by injection under a nitrogen atmosphere to a cold solution (ice bath) of the acid 3b (280 mg, 0.54 mmol) in tetrahydrofuran (THF) (3 mL). The resulting solution was stirred at 0.degree. C. for fifteen (15) minutes. Water was added, and the mixture was extracted several times with ether. The ether layer was dried and evaporated to give the crude product. The product was further purified by silica gel column with ether-petroleum ether (1:1) as eluent. The solid thus obtained (140 mg, 56%) was crystallized from acetonitrile to give the acid 3a: mp 112°-114° C. (sintering); [∀]_{D} −275.degree. (c 3.8 mg/mL, CHCl₃); IR 8ₘₐₓ (Nujol) 1680 cm⁻¹ and a broad band in the 3100-3600 cm⁻¹ region; ¹H NMR (CDCl₃) Δ 3.82 (1H, br d, J=18 Hz, C-2 eq H), 6.22 (1H, d, J=18 Hz, C-2 eq H), 6.22 (1H, d, J=1.5 Hz, arom), 6.38 (1H, d, J=1.5 Hz, arom), 7.16 (1H, m, C-6, H); m/z 400(M); HRMS calculated for C₂₅H₃₆O₄ 400.2613, found 400.2592.

e. Synthesis of (3R,4R)-Δ⁸-THC-DMH-11-oic Acid Acetate (3c)

A solution of acid 3a (100 mg, 0.25 mmol) in pyridine (2 mL) and acetic anhydride (1 mL) was stirred overnight at room temperature. Water (5 mL) was added to hydrolyze any mixed anhydride formed. The mixture was stirred for two (2) hours and then partitioned between water and ether. The ether layer was washed with dilute HCl (to remove the pyridine) and water. The organic layer was dried and evaporated. Pure product was obtained by preparative TLC (eluent ether-petroleum ether, 60:40) and crystallization from pentane. The acetate 3c, 65 mg, melts at 120°-122° C.: [∀]_{D} −265° (c 9.0 mg/mL, CHCl₃); IR 8ₘₐₓ (Nujol) 1760 cm⁻¹ and a broad band in the 3100-3600 cm⁻¹ region; ¹H NMR (CDCl₃) Δ 2.30 (3H, s, OCOCH₃), 3.38 (1H, br d, J=19 Hz, C-2 eq H), 6.56 (1H, d, J=1.5 Hz, atom), 6.68 (1H, d, J=1.5 Hz, arom), 7.18 (1H, m, C-6, H); HRMS calculated for C₂₇H₃₈O₄ 442.2719, found 442.2691.

f. Preparation of Capsules

A large number of unit capsules are prepared by filling standard two-piece hard gelatin capsules each with the desired amount of the powdered active ingredient as described above, 150 milligrams of lactose, 50 milligrams of cellulose, and 6 milligrams magnesium stearate. The capsules may also be prepared to include existing compounds useful in treating nausea.

g. Preparation of Soft Gelatin Capsules

A mixture of active ingredient in a digestible oil such as soybean oil, lecithin, cottonseed oil or olive oil is prepared and injected by means of a positive displacement pump into gelatin to form soft gelatin capsules containing the desired amount of the active ingredient. The capsules are washed and dried for packaging. The soft gelatin capsules may also be prepared to include existing compounds useful in treating nausea.

h. Preparation of IV Formulation

A formulation suitable for intravenous administration is prepared by dissolving the desired amount of the active ingredient as described above in a suitable volume of saline. The formulation may also be prepared to include existing compounds useful in treating nausea, and/or anticholineric agents. Because the active ingredient may be relatively insoluble in water, it can be advantageously incorporated into liposomes.

### 5. Conclusion

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating mammals suffering from nausea using a compound having Formula II 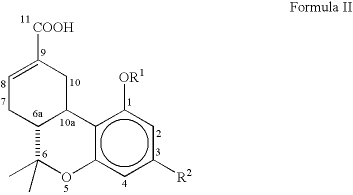
wherein R¹ is hydrogen, —COCH₃ or —COCH₂CH₃; R² is a branched C₅-C₁₂ alkyl group, which may optionally have a terminal aromatic ring, or optionally a branched —OCHCH₃(CH₂)ₘ alkyl group, which may have a terminal aromatic ring, wherein m is 0 to 7, or a pharmaceutically acceptable salt, ester, or solvate thereof, the method comprising:
identifying a mammal suffering from or suspected of suffering from nausea; and
administering to the mammal an effective amount of the compound of Formula II.

2. The method of claim 1, wherein R¹ is hydrogen.

3. The method of claim 2, wherein R² is a C₉ alkyl.

4. The method of claim 3, wherein the C₉ alkyl is a branched alkyl.

5. The method of claim 4, wherein the branched alkyl is 1,1-dimethylheptyl.

6. The method of claim 1, wherein R² is a C₉ alkyl.

7. The method of claim 6, wherein the C₉ alkyl is a branched alkyl.

8. The method of claim 7, wherein the branched alkyl is 1,1-dimethylheptyl.

9. The method of claim 1, wherein the mammal is a human.

10. The method of claim 1, wherein the compound is administered orally.

11. The method of claim 1, wherein the compound is administered intravenously.

12. The method of claim 1, wherein the compound is administered via an implant.

13. The method of claim 12, wherein the implant provides slow release of the compound.

14. The method of claim 1, wherein the compound is administered in a tablet.

15. A pharmaceutical composition for use in treating nausea in a mammal, comprising:
a compound having Formula II 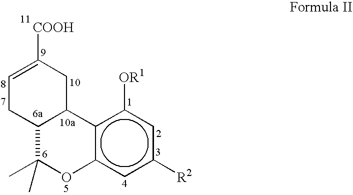
wherein R¹ is hydrogen, —COCH₃ or —COCH₂CH₃; R² is a branched C₅-C₁₂ alkyl group, which may optionally have a terminal aromatic ring, or optionally a branched —OCHCH₃(CH₂)ₘ alkyl group, which may have a terminal aromatic ring, wherein m is 0 to 7, or a pharmaceutically acceptable salt, ester, or solvate thereof.

16. The pharmaceutical composition of claim 15, further comprising an anticholinergic agent selected from the group consisting of anisotropine, aprophen, artane, atropine, belladonna, benactyzine, benztropine, clidinium, dicyclomine, glycopyrrolate, homatropine, hyoscyamine, isopropamide, mepenzolate, methantheline, methscopolamine, oxybutynin, oxyphencyclimine, propantheline, scopolamine, terodiline, tridihexethyl, trihexyphenidyl, and trospium.

17. The pharmaceutical composition of claim 15, further comprising an agent useful in relieving symptoms of nausea selected from the group consisting of meclizine, Emetrol®, hydroxizine (Atarax® Vistaril®) and dimenhydrinate (Dramamine®).

18. The pharmaceutical composition of claim 15, wherein said pharmaceutical composition is formulated for oral administration.

19. The pharmaceutical composition of claim 15, wherein said pharmaceutical composition is formulated for intravenous administration.

20. A pharmaceutical composition for use in treating nausea in a mammal, comprising:
a compound having Formula III 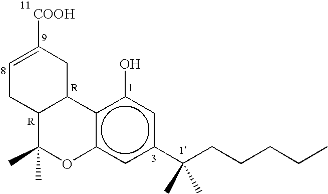
or a pharmaceutically acceptable salt, ester, or solvate thereof.
